SEQUENCE LISTING (4 sequences)

SEQ ID NO: 1 - DNA, 30 bp, Artificial
  Murine Primer
aaaggatccaaaaagcggccaaagcctgga

SEQ ID NO: 2 - DNA, 31 bp, Artificial
  Murine Primer
cccaagcttaggatcttctcccgtcgtaata

SEQ ID NO: 3 - DNA, 41 bp, Artificial
  Murine Primer
gaaaacctgtattttcagggcaaaaagcggccaaagcctgg

SEQ ID NO: 4 - DNA, 41 bp, Artificial
  Murine Primer
ccaggctttggccgctttttgccctgaaaatacaagttttc

(19) United States
(12) Patent Application Publication
(10) Pub. No.: US 2011/0183421 A1
(43) Pub. Date: Jul. 28, 2011

(54) COMPOSITION AND METHOD FOR CONVERTING A NON-PATHOGENIC PRION PROTEIN INTO A PATHOGENIC CONFORMATION AND USES THEREOF

(76) Inventors: Jiyan Ma, Columbus, OH (US); Fei Wang, (US); Xinhe Wang, Columbus, OH (US)

(21) Appl. No.: 13/015,135

(22) Filed: Jan. 27, 2011

Related U.S. Application Data
(60) Provisional application No. 61/298,806, filed on Jan. 27, 2010.

(51) Int. Cl. G01N 1/28; C07H 21/00; C07H 21/02; C07F 9/113

(52) U.S. Cl. 436/17; 536/23.1; 554/79; 436/86

(57) ABSTRACT

Described herein is a composition for converting a non-pathogenic prion protein (“PrP^{N}”) into a prion protein in a pathogenic conformation (“PrP^{Path}”) comprising a lipid and a polyanion. Also described are methods of (1) using the composition to convert a PrP^{N} to a PrP^{Path}, (2) identifying a potential therapeutic substance affecting PrP^{Path}, and (3) diagnosing PrP^{Path} infection in a subject using the composition and at least one cycle of protein misfolding cyclic amplification.

### RELATED APPLICATION

This application claims the benefit of U.S. Provisional Application Ser. No. 61/298,806, filed Jan. 27, 2010, the disclosure of which is expressly incorporated by reference herein in its entirety.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of Grant No. R01NS060729 awarded by the National Institutes of Health.

### FIELD OF THE INVENTION

The present invention relates generally to methods and compositions promoting the conversion of a prion protein (PrP) into a pathogenic conformation, and more specifically to methods and compositions directed to the diagnosis of a prion disease with a composition that promotes the conversion of a prion protein into its pathogenic conformation.

### BACKGROUND

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Transmissible spongiform encephalopathies (TSEs or prion disease) are catastrophic infectious neurodegenerative disorders that are difficult to diagnose at early stages of infection. Compositions and diagnostic methods are needed to improve the efficiency of diagnosing this disease.

The prion hypothesis for TSEs proposes that the infectious agent is an aberrant conformational isoform of the normal cytosolic prion protein (PrP^{C}), a GPI (glycosylphosphatidylinositol)-anchored glycoprotein. By virtue of its self-perpetuating characteristic, the aberrant pathogenic prion protein isoform (PrP^{Path}) converts host normal PrP^{C} into the PrP^{Path} conformation and leads to neurodegeneration. Despite strong evidence supporting the prion hypothesis, a crucial prediction derived from the hypothesis, that an infectious prion can be generated with bacterially expressed recombinant PrP (rPrP), remains unfulfilled, leaving lingering doubts about the prion hypothesis.

Recombinant PrP has been folded into various forms similar to PrP^{Path}, but none of them fully recapitulates the characteristics of the infectious agent. The amyloid fiber of an rPrP fragment (rPrP89-230) causes prion disease in transgenic mice over-expressing PrP89-230, but a prolonged incubation time in mice over-expressing PrP has led to uncertainty about whether the infectivity is indeed derived from rPrP89-230 amyloid fibers. The difficulty in creating a recombinant prion is likely due to the lack of proper facilitating factors. Compositions that facilitate the conversion of rPrP into its pathogenic form in the presence of PrP^{Path} are needed to facilitate the use of rPrP in diagnosing prion disease.

### SUMMARY

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

Polyanions, particularly RNA, have been observed to facilitate normal PrP^{C} conversion into PrP^{Path} and promote de novo prion formation. GPI-anchored PrP^{C} is in the vicinity of lipid membranes and the interfacial lipid bilayer region strongly influences protein structure. And so we investigated lipid as a potential facilitating factor for the conversion of normal PrP^{C} into PrP^{Path}. Encouraged by the findings that lipid interaction converts rPrP to a PrP^{Path}-like form, we applied protein misfolding cyclic amplification (PMCA) to study rPrP conversion in the presence of both lipid and RNA.

Thus, one aspect of the present invention provides a composition, referred to herein as a conversion substrate, for converting a non-pathogenic prion protein (PrP^{N}), such as rPrP or normal PrP^{C}, into a prion protein in a pathogenic conformation (PrP^{Path}). In particular, an embodiment of the composition includes a lipid and/or a polyanion. Further, the lipid can be a synthetic phospholipid such as 1-palmitoyl-2-oleoyl phosphatidylglycerol and the polyanion can be a nucleic acid such as total RNA isolated from a tissue such as mouse liver or synthetic polyribonucleic acids.

Another aspect of the present invention provides a method of converting a PrP^{N} into a PrP^{Path} by mixing a PrP^{N} with a conversion substrate including a lipid and/or a polyanion and conducting at least one cycle of protein misfolding cyclic amplification (PMCA) on the mixture. Further, the conversion substrate may be seeded with a naturally occurring, model animal adapted, or synthetic PrP^{Path}.

Another aspect of the present invention provides a method of identifying a potential therapeutic substance affecting a PrP^{Path} comprising mixing a potential therapeutic substance with a PrP^{N} and a conversion substrate, wherein the conversion substrate includes a lipid and/or a polyanion, conducting at least one cycle of PMCA on the mixture, and evaluating the mixture to determine if the potential therapeutic substance affected the conversion of the PrP^{N} into a PrP^{Path}. Further, the mixture of the potential therapeutic substance with the PrP^{N} and the conversion substrate may be seeded with a naturally occurring, model animal adapted, or synthetic PrP^{Path}.

Another aspect of the present invention provides a method of diagnosing PrP^{Path} infection in a subject comprising mixing a sample of tissue, blood, or body fluid from a subject with a PrP^{N} and a conversion substrate wherein the conversion substrate includes a lipid and/or a polyanion, conducting at least one cycle of protein misfolding cyclic amplification on the mixture of the sample, the PrP^{N} and the conversion substrate, and evaluating the mixture to determine if the sample functioned to seed the conversion of the PrP^{N} into a PrP^{Path}.

### BRIEF DESCRIPTION OF THE DRAWINGS

The accompanying drawings, which are incorporated in and constitute a part of this specification, illustrate embodiments of the invention and, together with the general description of the invention given above and the detailed description of the embodiments given below, serve to explain the principles of the present invention.

FIG. 1 is schematic illustrating the application of multiple rounds of amplification via PMCA.

FIG. 2 is a representative immunoblot demonstrating the conversion of rPrP to rPrP-res after multiple rounds of protein misfolding cyclic amplification (PMCA) in accordance with the invention.

FIG. 3 is a representative immunoblot of PK-resistant PrP (rPrP-res) following multiple PMCA reactions of undigested amplification products from FIG. 2.

FIG. 4 is an illustrative blot showing de novo rPrP-res formation.

FIG. 5A is a representative immunoblot of brain homogenate with or without PrP-res seeding after proteinase K digestion.

FIG. 5B is a representative immunoblot of a in vitro generated rPrP-res after proteinase K digestion.

FIG. 6 is a representative immunoblot of PMCA product that was separated into supernatant or pellet.

FIG. 7 is a representative immunoblot of SN56 cell lysate after infection with rPrP-res.

FIG. 8 is a representative n immunoblot analysis of PrP-res formation.

FIG. 9A is a representative photograph of an immunoblot illustrating the presence of PrP^{Path}.

FIG. 9B are photographs demonstrating the neurological symptoms from mice inoculated with rPrP-res.

FIG. 10 is a graph plotting the survival of mice injected with rPrP-res.

FIG. 11 is photomicrograph of a histosection of a mouse brain inoculated with rPrP-res.

FIG. 12 shows a series of photomicrographs of histosection of a mouse brain inoculated with rPrP-res.

FIG. 13 includes panels A-F showing a series of photomicrographs of histological sections from brains of control mice and mice inoculated with rPrP-res

FIG. 14 is a series of photomicrographs of histosections of a mouse brain inoculated with rPrP-res and a graph illustrating the extent of vacuolization in the mouse brains.

FIG. 15 includes panels A-D, which are photomicrographs of histological sections from brains of control mice and mice inoculated with rPrP-res.

FIG. 16 are photomicrographs of paraffin embedded tissue blot of brains of control mice and mice inoculated with rPrP-res.

FIG. 17 is a representative blot demonstrating the presence of PrP^{Path}.

FIG. 18A is a representative immunoblot of homogenates from the brains of mice inoculated with rPrP-res.

FIG. 18B is a representative immunoblot of homogenates from the brains of mice inoculated with rPrP-res.

FIG. 19 is a graph plotting the survival of a second round of infected mice.

FIG. 20 is a representative blot demonstrating the presence of PrP^{Path}.

FIG. 21 shows a series of photomicrographs of histosections of a mouse brain that received a second round of infection and a brain from a control mouse.

FIG. 22 is a silver staining blot of purified rPrP.

FIG. 23 is a representative immunoblot of a rPrP-res propagated with conversion substrate having synthetic poly(A) RNA as the polyanion.

### DETAILED DESCRIPTION

One or more specific embodiments of the present invention will be described below. In an effort to provide a concise description of these embodiments, all features of an actual implementation may not be described in the specification. It should be appreciated that in the development of any such actual implementation, as in any project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking for those of ordinary skill having the benefit of this disclosure.

Certain aspects of the present invention include compositions that promote the conversion of PrP^{N} into PrP^{Path}. Examples of PrP^{N} include normal prion protein found in a cell, i.e., PrP^{C} and recombinant prion protein in the non-pathological conformation grown in a bacterial cell (rPrP). The composition, also referred to herein as a conversion substrate, include a lipid and/or a polyanion. The lipid and/or polyanion are included in a sufficient amount to promote the conversion of PrP^{N} to PrP^{Path}. The composition may further include at least one buffer.

A lipid useful in embodiments of the invention includes at least one of an anionic lipid, a cationic lipid, a zwitterionic lipid or a neutral lipid. The lipid may be a phospholipid. The lipid may also be a synthetic lipid. In one embodiment, the lipid is a synthetic anionic phospholipid, such as 1-palmitoyl-2-oleoylphosphatidylglycerol (POPG). In addition to POPG, the lipid may also include 1-palmitoyl-2-oleoyl-sn-glycero-3-phosphocholine (POPC), 1-palmitoyl-2-arachidonoyl-sn-glycero-3-phosphocholine (PAPC), 1-palmitoyl-2-arachidonoyl-sn-glycero-3-phosphoglycerol (PAPG) or lipids extracted from a tissue, such as total lipids extracted from an animal's brain or other organ. The lipid may be a single type of lipid or a combination of lipid types.

The lipid component of the conversion substrate may be prepared by drying the lipid, then hydrating the dried lipid, and finally by mixing the hydrated lipid until clear. In one embodiment, a lipid dissolved in a solvent, such as POPG in chloroform, is dried, such as under a stream of nitrogen at 42° C. The dried lipid is then hydrated, such as in a 20 mM Tris-HCl buffer (pH 7.4). The final lipid concentration of the rehydrated lipid may be in a range of about 1 ng/ml to about 10 mg/ml, or from about 1 ng/ml to about 100 ng/ml, or from about 1 ng/ml to about 10 ng/ml. In one embodiment, the final lipid concentration is about 5 ng/ml. The hydrated lipids are mixed, such as by vortexing, sonicating, and/or by extrusion, until the solution appears to be clear, i.e., the lipid no longer appears as distinct particles in the solution when observed with the naked eye of a normal observer.

A polyanion useful in embodiments of the invention includes a nucleic acid, a protein, a carbohydrate, peptide, a proteoglycan and a polyphosphate. The polyanion may be natural or synthetic. The polyanion may also be modified from its original state. In one embodiment, the polyanion is a ribonucleic acid (RNA). The polyanion may be a single type of polyanion or a combination of polyanion types.

In one embodiment, the polyanion component of the conversion substrate is isolated from a cell or a tissue. For example, RNA may be isolated from a tissue by homogenizing the tissue in a RNA isolation reagent, and extracting the RNA from the homogenized tissue with a hydrophobic organic solvent such as chloroform, precipitating the extracted RNA with an alcohol, and dissolving the precipitated RNA in RNAse free water.

Buffers useful in embodiments of the invention include Tris-HCl buffer, phosphate buffered saline, and TN buffer. In one embodiment, the buffer includes deionized water, a detergent, such as Triton X-100, and 10× TN buffer (1.5 M NaCl, 100 mM Tris-HCl, pH 7.5). The pH of the buffer may be in a range of about 6.0 to about 8.0. In another embodiment, the pH of the buffer is in a range of about 7.0 to about 7.8. In another embodiment, the pH of the buffer is in a range of about 7.2 to about 7.6. In another embodiment, the pH of the buffer is in a range of about 7.4 to about 7.5. In another embodiment, the buffer is at a physiological pH.

In one embodiment, the conversion substrate is prepared by first mixing the PrP^{N} with the lipid component. This mixture is then mixed with a buffer component. The polyanion component is then added to the mixture.

The protein misfolding cyclic amplification (”PMCA″) protocol includes alternating cycles of sonication and incubation periods, such as may be conducted with a microplate sonicator. For example, the conversion substrate with PrP^{N} is added to the wells of a microplate and sonicated at a controlled temperature for a short period of time, such as about 30 seconds. In some embodiments, the period of time may range between about 10 seconds and about 5 minutes or between about 20 seconds and about 1 minute or between about 20 seconds and about 45 seconds. After sonication, the mixture is allowed to incubate without sonication for a period of time that is longer than the period time for the sonication. In one embodiment, the mixture incubates for a period of time that ranges from about 5 minutes to about 1 hour. However, it is understood that the mixture may incubate for different periods of time that range from about 15 minutes to about 45 minutes or from about 20 minutes to about 30 minutes or for about 25 minutes. Each sonication/incubation cycle is repeated numerous times in a round, such as about 10 times. However, it is understood that round may be comprised of repeating the sonication/incubation cycle at least about 15 times, or about 20 times or more, or about 30 times or more, or about 40 times or more, or about 45 times or more, or about 48 times. The sonication/incubation cycles are typically maintained at a physiological temperature range of around 37° C. However, it is understood that the temperature range may be optimized to vary at different points in the cycle between about 20° C. and about 90° C., or between about 25° C. and about 50° C. The level of sonication may vary, but should be sufficient to promote conversion of the PrP^{N} to the PrP^{Path}.

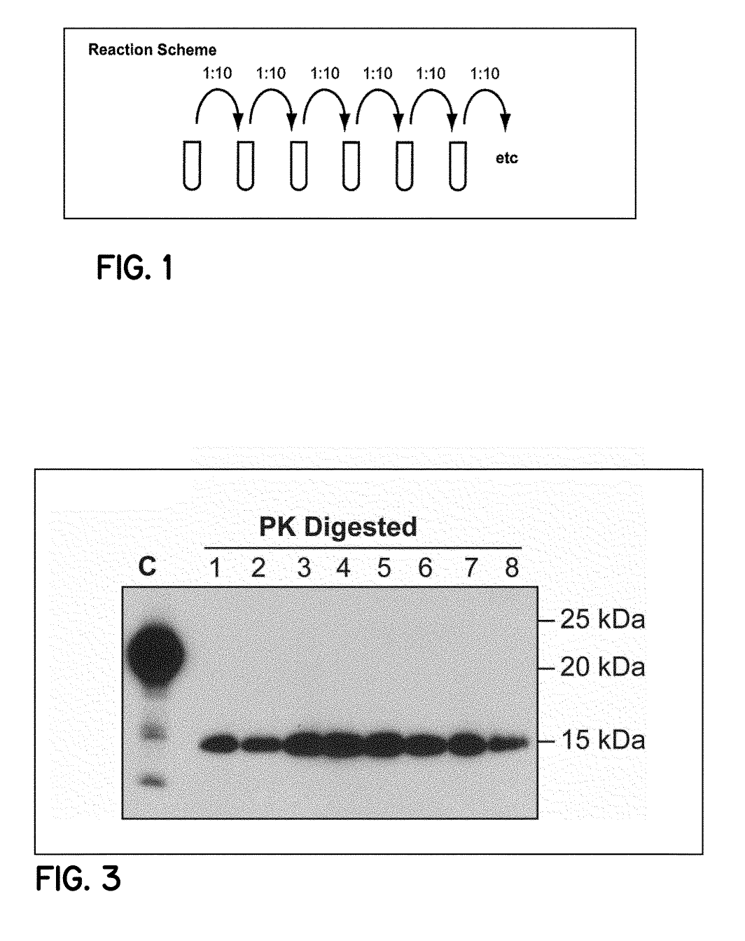

The conversion substrate and PrP^{N} mixture may be subjected to multiple rounds of amplification. In one embodiment, a portion of the mixture that has already been subjected to at least one round of amplification is mixed with fresh conversion substrate and subjected to an additional round of amplification. This is referred to as serial amplification and as illustrated in FIG. 1 each round includes 48 cycles of 30 seconds of sonication followed by 29.5 minutes of incubation. At the end of each round, 1/10^{th} of the reaction mixture is transferred to a new tube of conversion substrate to start a new PMCA round. Serial amplification may include as many rounds a necessary to achieve the desired level of amplification. In one embodiment, serial amplification includes about 5 or more rounds, or about 10 or more rounds, or about 15 or more rounds, about 20 or more rounds, or about 25 or more rounds, or about 30 or more rounds, or about 35 or more rounds, or about 40 or more rounds, or about 45 or more rounds, or about 50 or more rounds. In one embodiment, serial amplification includes about 1 to about 100 rounds of amplification.

One aspect of the invention described herein is the use of the prion conversion methods to identify potential therapeutic substances, such as prion protein mutants and small molecules including peptides that might affect the formation of PrP^{Path} and infectivity. The therapeutic substances may, for example, interfere with the conversion of a PrP^{N} into a PrP^{Path}, interfere with infectivity of the PrP^{Path}, or prevent and/or disrupt the interaction of the prion protein with a lipid and/or a polyanion. In one embodiment, the method includes mixing a potential therapeutic substance with a PrP^{N} and the conversion substrate. In an alternative embodiment, the method includes mixing a potential therapeutic substance with a mixture that includes PrP^{N} in the conversion and seeded with PrP^{Path}. At least one round of the PMCA protocol is conducted on the selected mixture. The mixture is then evaluated to determine if the potential therapeutic substance affected the conversion of the PrP^{N} into a PrP^{Path}. In one embodiment, evaluating the mixture includes at least one of determining the presence of prion protein in a pathogenic confirmation, i.e., PrP^{Path}, the conversion rate of prion protein into a pathogenic confirmation, i.e., PrP^{N} conversion to PrP^{Path}, and comparing the results obtained with the mixture including the potential therapeutic substance with a control mixture lacking the potential therapeutic substance.

One aspect of the invention described herein is use of the method to diagnose PrP^{Path} infection in a subject. The diagnostic method includes mixing a sample of tissue, blood, or body fluid from a subject (with or without processing) with PrP^{N} and conversion substrate. The mixture is then subjected to at least one round of the PMCA protocol. The mixture is then evaluated to determine if the sample functioned to seed the conversion of the PrP^{N} into a PrP^{Path}. Levels of severity of infection can be determined by comparing the amount of amplification that results from an infected tissue with a level of amplification the results from a series of controls having known concentrations of PrP^{Path}.

Various aspects of the present invention are further illustrated by the following non-limiting Example.

### EXAMPLE

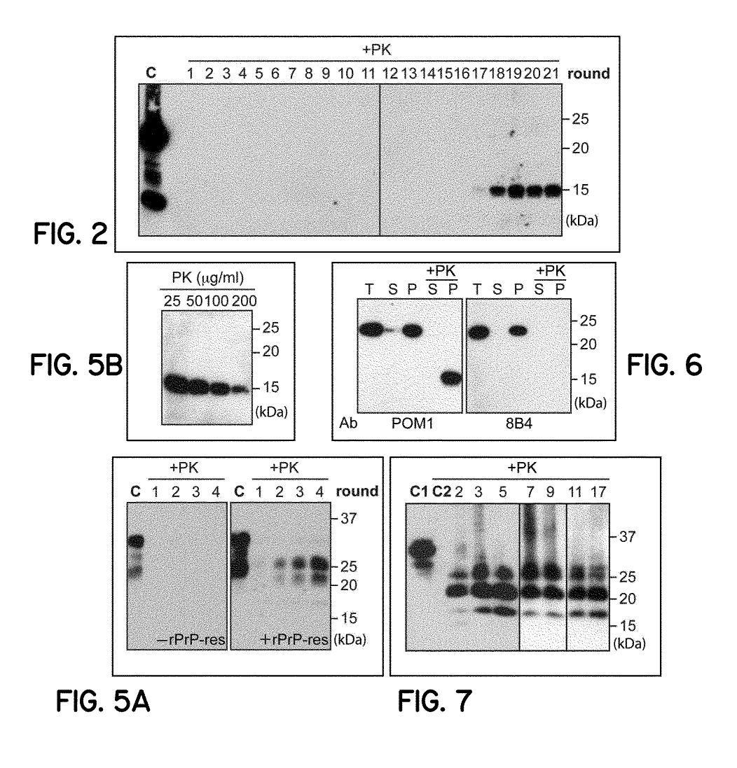

Using a serial PMCA protocol, we tested several conversion substrates in which rPrP was mixed with various combinations of lipids and/or total RNA isolated from normal mouse liver. In the presence of synthetic anionic phospholipid POPG (1-palmitoyl-2-oleoylphosphatidylglycerol) and RNA, a 15 kDa proteinase K (PK)-resistant band was detected after 17 rounds of PMCA (FIG. 2). Each round of PMCA included 48 cycles of sonication (0.5 minutes) and incubation (29.5 minutes). At the end of each round, 1/10^{th} of the reaction mixture was transferred to a tube containing fresh reaction substrate to start a new round. The PMCA products were then digested with 25 μg/ml proteinase K. Once formed, the PK-resistant rPrP (rPrP-res), which serves as a model for PrP^{Path}, was able to serially propagate (FIGS. 2A and 3). For FIG. 3, the undigested PMCA products from rounds 19 and 20 in FIG. 2A were pooled together and used to seed 8 reactions for another PMCA round. The PK-resistant PrP was detected by immunoblot analysis with POM1 anti-PrP antibody. The control (c) is undigested rPrP.

The same procedure was repeated several times and, in our hands, the overall efficiency of de novo rPrP-res formation was around 20

STATEMENT REGARDING FEDERALLY SPONSORED RESEARCH OR DEVELOPMENT

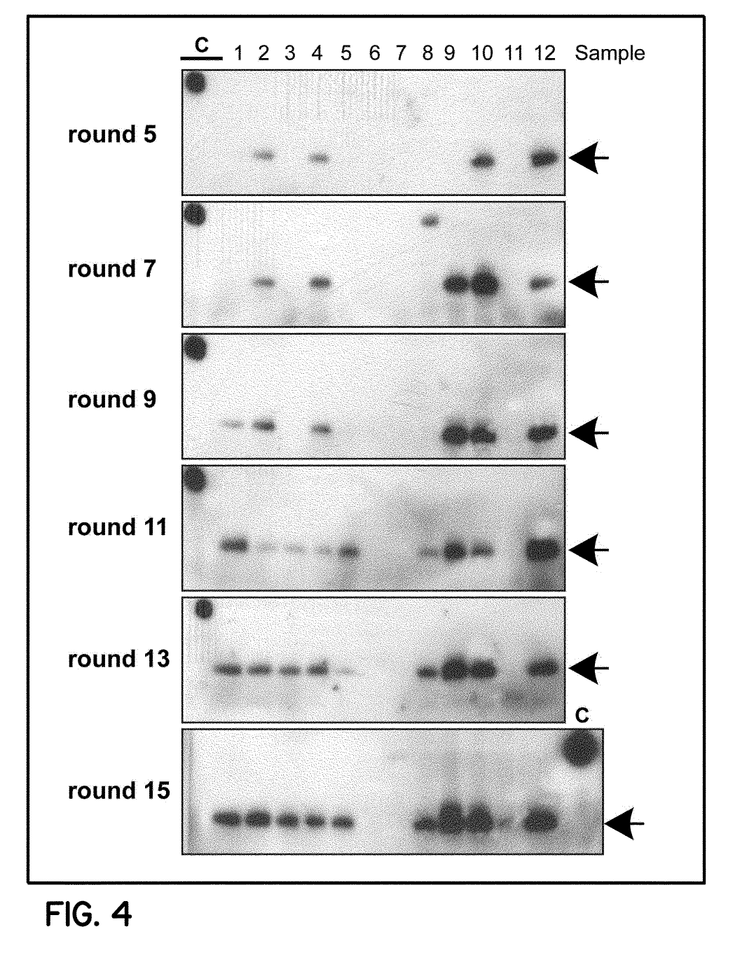

The U.S. Government has a paid-up license in this invention and the right in limited circumstances to require the patent owner to license others on reasonable terms as provided for by the terms of Grant No. R01NS060729 awarded by the National Institutes of Health.% (FIG. 4). The de novo rPrP-res formation in this experiment was performed 6 times and the success rate for each experiment was 1/1; 0/32; 0/16; 3/16; 5/12; and 9/12. Overall, there were 18 successful de novo rPrP-res formation out of 89 samples. For this experiment, the substrates were prepared in a new lab with new pipettes, and the PMCA was carried out in a new sonicator connected with a new circulating water bath. None of which had been exposed to prion. The de novo formation of PK-resistant PrP-res (pointed to by the arrows) in the indicated PMCA rounds was detected by immunoblot analysis with POM1 antibody. The control (C) is undigested rPrP.

As seen in FIG. 5A, serial PK-digestion of rPrP-res revealed that the PK-resistant band was detectable after 200 μg/ml PK digestion (PK:rPrP molar ratio>50:1). For this study, normal mouse brain homogenate was used as the substrate, and PMCA was performed with or without rPrP-res seed. The product was digested with 100 g/ml proteinase K. The control (C) is undigested normal mouse brain homogenate. As seen in FIG. 5B, serial digestion of PMCA products reduced the amount of staining observed in the samples.

After centrifugation, the rPrP-res was only detected in the pellet fraction (FIG. 6), indicating that rPrP-res was aggregated. For these data, the PMCA product was separated into supernatant(s) and pellet(s) by a 1 hour 100,000 g centrifugation at 4° C. The 15 kDa PK-resistant band was not recognized by the 8B4 antibody that detects an N-terminal epitope of PrP (FIG. 6), revealing that the rPrP-res contained a C-terminal PK-resistant core. Thus, similar to PrP^{Path}, rPrP-res is aggregated, PK-resistant, and contains a C-terminal PK-resistant core.

Next, we performed PMCA and cell culture analyses to determine whether rPrP-res could seed glycosylated and GPI-anchored endogenous PrP^{C}. With normal mouse brain homogenate as substrate, PMCA was carried out with or without rPrP-res seed. The PK-resistant endogenous PrP, demonstrated by higher molecular weights of glycosylated PrP, was detected in samples seeded with rPrP-res (FIG. 5). Whereas in reactions without rPrP-res seed, no PK-resistant PrP was detected, ruling out de novo PrP-res formation or insufficient PK digestion. The cell infection assay was performed on SN56 cells, a murine neuronal cell line susceptible to prion infection. Endogenous PrP^{C} in SN56 cells was glycosylated and sensitive to PK digestion (FIG. 7). After rPrP-res infection, the PK-resistant endogenous PrP was detected in passage 2 cells and remained detectable after 17 passages (FIG. 7). The numbers at the top of the columns indicates the number of cell passages. Control 1 (C1) was undigested SN56 lysate and control 2 (C2) is the pellet of PK-digested, uninfected SN56 cell lysates.

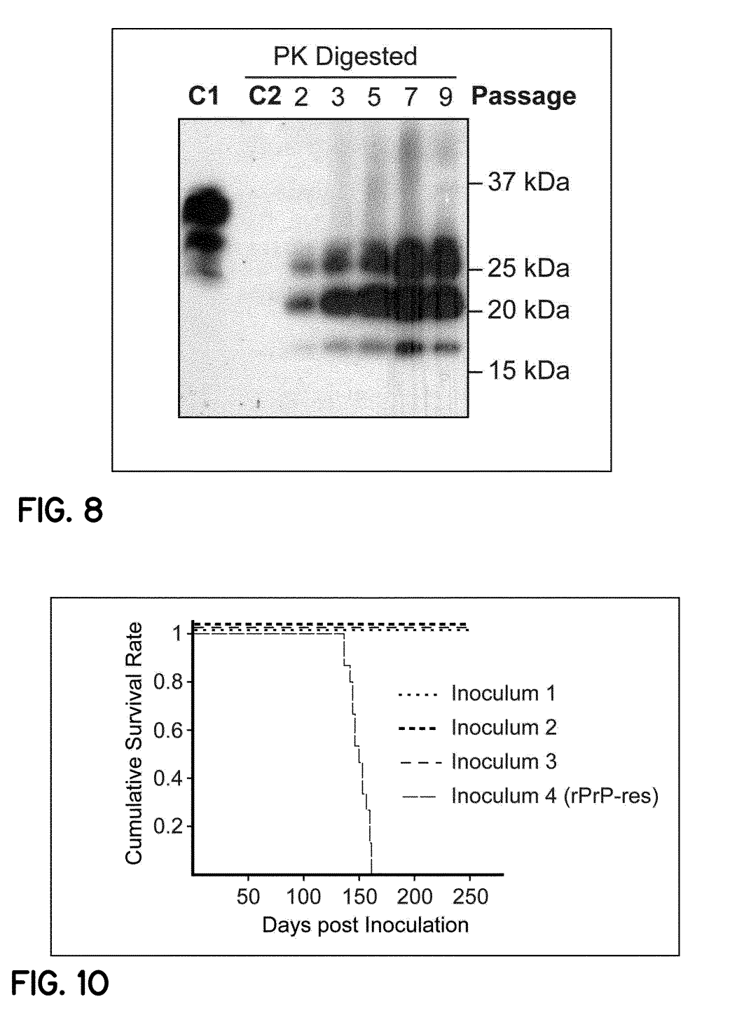

A similar experiment revealed that the rPrP-res converted normal mouse brain homogenate (FIG. 5) could infect SN56 cells as well (FIG. 8). Thus, rPrP-res was able to propagate its PK-resistant conformation to endogenous PrP^{C}.

To determine whether rPrP-res was capable of causing bona fide prion disease, we infected 8-week-old female CD-1 mice by intracerebral injection. The rPrP-res (inoculum 4) was prepared by propagating rPrP-res through 24 rounds of PMCA. All PMCA products were pooled together and centrifuged through a sucrose cushion. The pellet was washed, resuspended, and used for inoculation. Three control inocula were used for animal study (see Table 1).

TABLE 1
Intracerebral inoculation of rPrP-res

| | | | Preparation for | Diseased/ | Survival |
|---|---|---|---|---|---|
| Inoculum | Component | Processing | injection | Inoculated | Time (dpi)† |
| 1 | Buffer + POPG + | Serial PMCA | Pelleting through | 0/15 | >360 |
| | RNA | | a sucrose cushion | | |
| | The amount of | | and washing | | |
| | each component | | twice with PBS | | |
| | equaled to that in | | | | |
| | the rPrP-res | | | | |
| | propagation | | | | |
| | reaction. | | | | |
| 2 | Buffer + POPG + | Incubated at | Pelleting through | 1*/14 | >360 |
| | RNA + rPrP | 37° C. without | a sucrose cushion | | (286*) |
| | The amount of | sonication | and washing | | |
| | each component | | twice with PBS | | |
| | equaled to that in | | | | |
| | the rPrP-res | | | | |
| | propagation | | | | |
| | reaction. | | | | |
| 3 | POPG + RNA + | No processing | No preparation | 0/5 | >360 |
| | rPrP | | | | |
| | The amount of | | | | |
| | each component | | | | |
| | equaled to that in | | | | |
| | the final pool of | | | | |
| | inoculum 4. | | | | |
| 4 | Buffer + POPG + | Serial PMCA | Pelleting through | 15/15 | 150 ± 2.2 |
| (rPrP-res) | RNA + rPrP + | | a sucrose cushion | | (mean ± SEM) |
| | rPrP-res seed | | and washing | | |
| | | | twice with PBS | | |

*One mouse died from an unrelated disease at 286 dpi. It had no neurological sign or weight loss.
†One mouse from each control groups was sacrificed at 275 dpi to serve as controls.

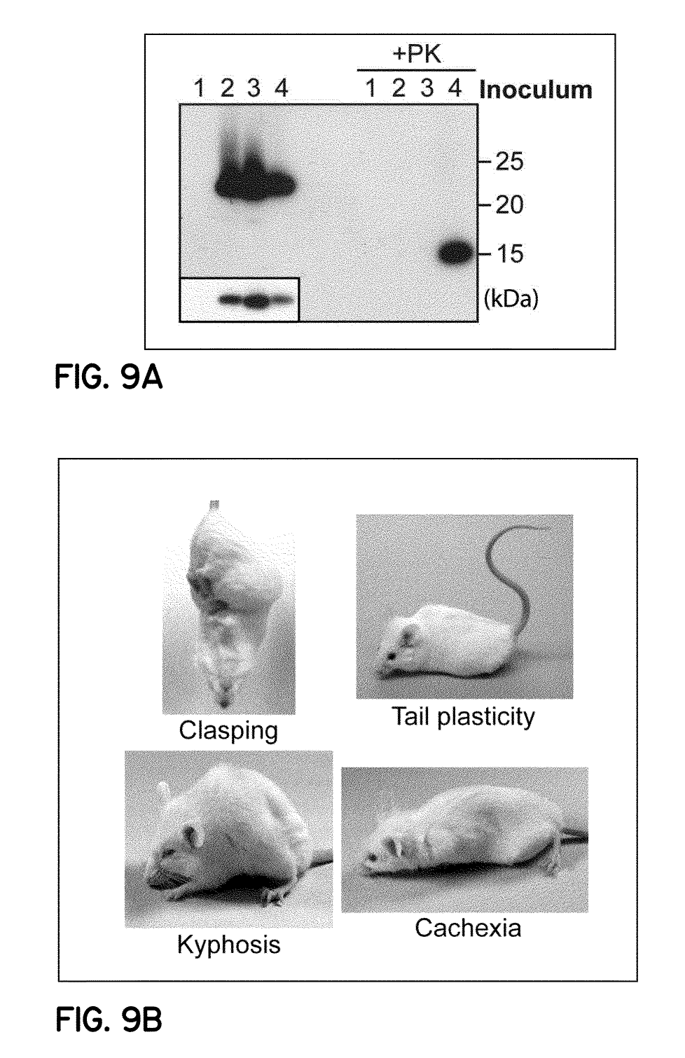

Inoculum 1, consisting of all the components used for rPrP-res propagation except for rPrP and rPrP-res seed, was subjected to the same treatments as inoculum 4. Inoculum 2, consisting of all the components of rPrP-res propagation except for rPrP-res seed, was incubated at 37° C. for 24 days without sonication, and subjected to the same pelleting and washing treatments. Omitting the sonication step prevented the de novo rPrP-res formation in this control sample, which was confirmed by the PK digestion analysis described below (FIG. 9A). Inoculum 3 was prepared by directly mixing rPrP, POPG, and RNA in the inoculum diluent. The amount of each component was equal to the total amount in the final pool of inoculum 4, ensuring that the result was not influenced by insufficient dosage of rPrP or any other component. The inoculation and animal care were carried out in an animal vivarium that had never been exposed to animals with prion disease.

After the injection, the remaining inocula were analyzed (FIG. 9A). Inoculum 1 did not contain PrP and, accordingly, no PrP was detected. Of three inocula that contained rPrP, inoculum 4 had the lowest amount of rPrP (FIG. 9A). However, the 15 kDa PK-resistant band was only detected in inoculum 4, verifying that it was the only inoculum containing rPrP-res. The insert shows a lighter exposure of undigested inocula.

Around 130 days post inoculation (dpi), all 15 rPrP-res inoculated mice developed clinical signs of prion disease. The earliest sign was clasping, an indication of neurological dysfunction (FIG. 9B). Soon after that, mice developed tail plasticity and akinesia, that is, mice remained stationary in response to external stimuli (FIG. 9B). The disease progressed rapidly and mice developed kyphosis, head twitching, mild ataxia, and eventually became cachexic and lethargic (FIG. 9B). The rPrP-res inoculated mice reached the terminal stage at 136-161 dpi and the average survival time was 150±2.2 days (mean±SEM) (Table 1 and FIG. 10). None of the mice injected with control inocula developed prion disease for more than 360 days. Thus, intracerebral rPrP-res injection caused neurodegenerative disorders in wild-type mice with infectivity specifically associated with the rPrP-res conformation.

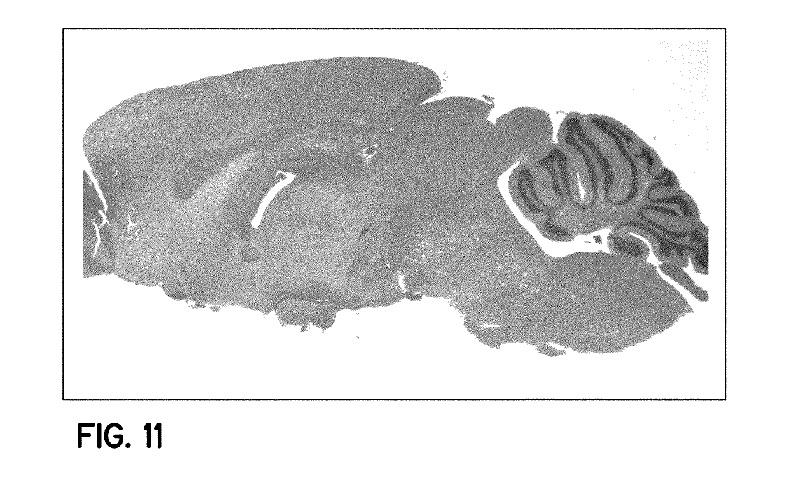

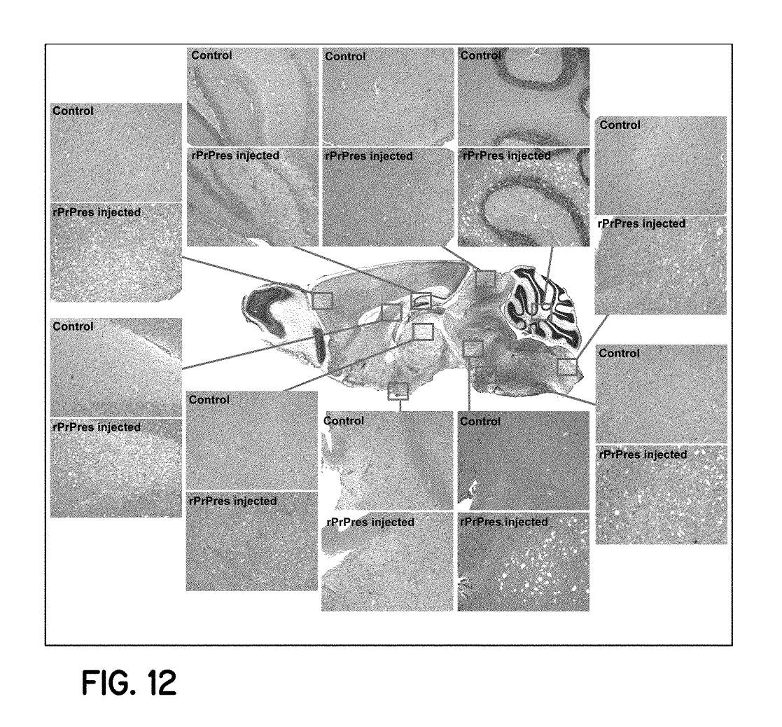

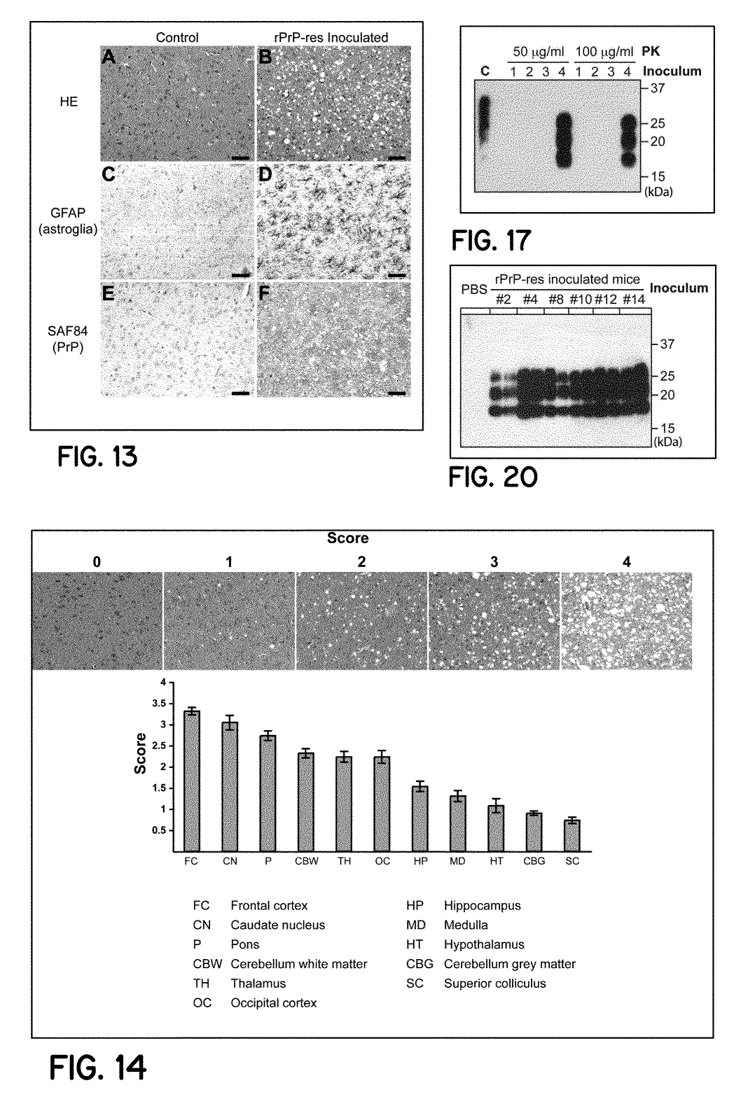

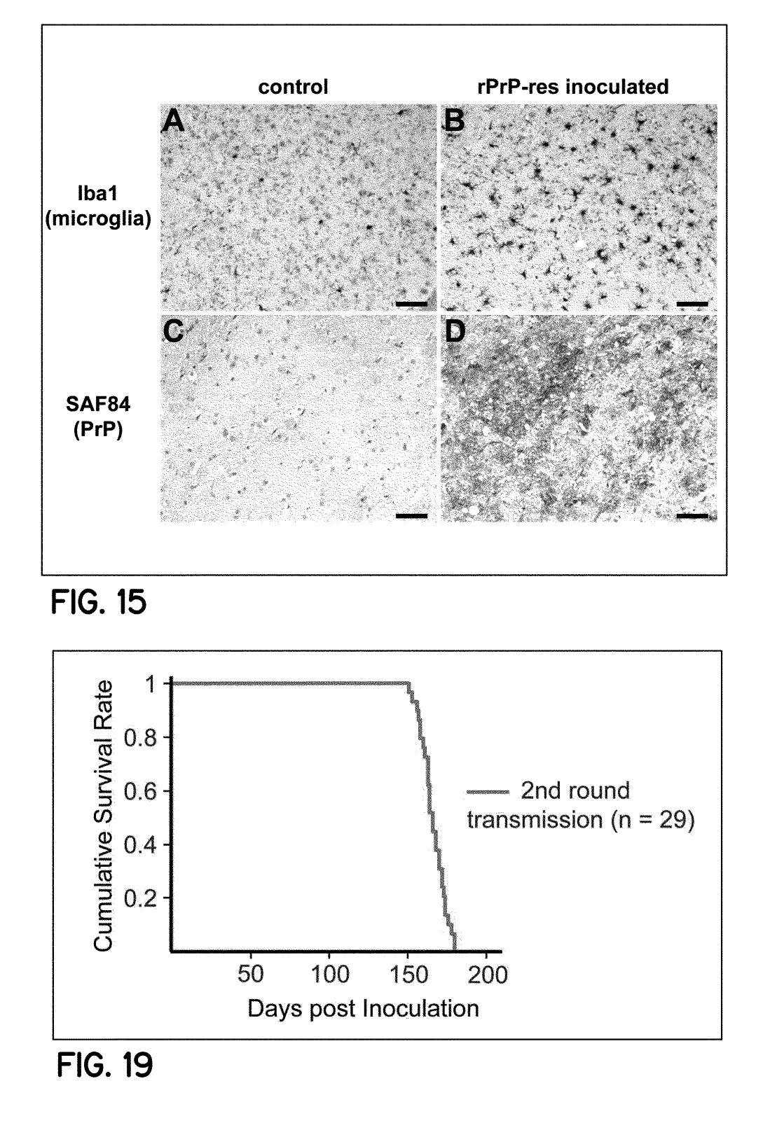

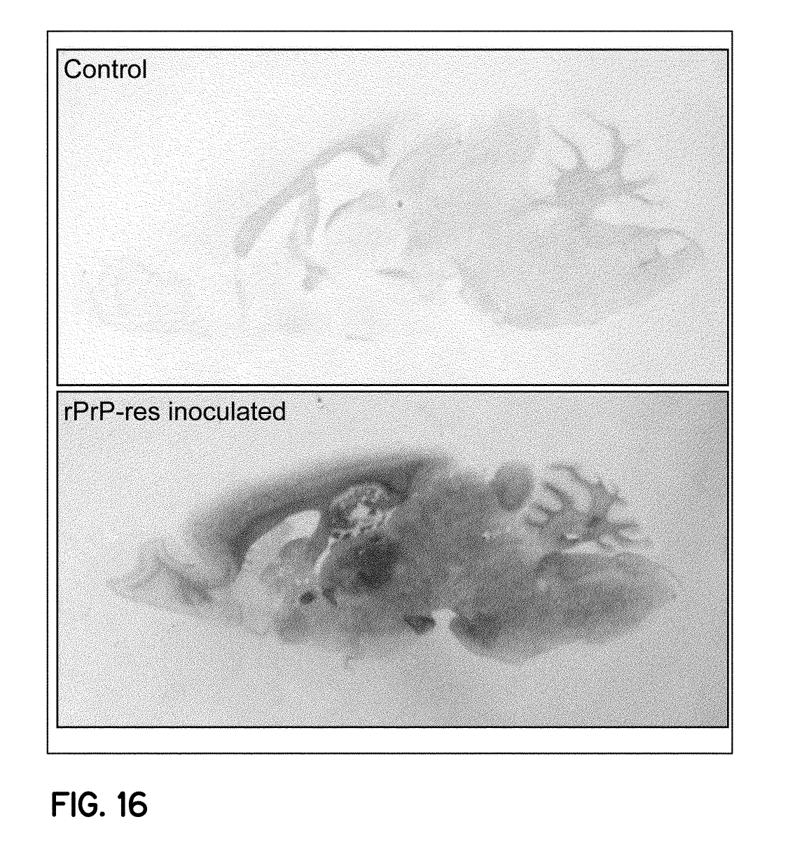

To ensure that every rPrP-res inoculated mouse received both pathological and biochemical analyses, each mouse brain was bisected sagittally and the half brain was subjected to histological or biochemical analysis. Severe spongiosis was detected in multiple brain regions (FIGS. 11 and 12). Dense small vacuoles were observed in the frontal cortex and caudate nucleus (FIG. 13 panel B and FIG. 12), while larger vacuoles were detected in the pons, midbrain (areas around raphe nuclei and periaqueductal gray) and cerebellar white matter (areas around cerebellar dentate and fastigial nuclei). Moderate spongiosis was present in the occipital cortex, thalamus, medulla, and hippocampus, whereas little spongiosis was detected in the superior or inferior colliculus, hypothalamus, or olfactory bulb (FIGS. 12 and 14). Prominent astrogliosis and microgliosis were detected in rPrP-res inoculated mouse brains (FIG. 13 panel D and FIG. 15). PrP immunohistochemistry revealed abnormal PrP deposition in a pattern similar to the diffuse synaptic accumulation (FIG. 13 panel F). The scale bar in FIG. 13 represents 50 μm. The densest PrP deposition was in thalamus (FIG. 15), which was supported by the paraffin-embedded tissue blot (PET blot) analysis (FIG. 16). FIG. 15 shows a immunohistochemical staining images of age- and sex-matched wild-type CD-1 control mice (A and C) and pPrP-res inoculated CD-1 mice (panels C and D). The sections shown in panels A and C were stain with an anti-Iba1 (ionized calcium-binding adaptor molecule 1) antibody. The sections shown in panels C and D were stain with SAF84 anti-PrP antibody, showing PrP deposition in thalamus. The immunohistochemical stains were counterstained with hematoxylin and the scale bar represents 50 μm. FIG. 16 shows images of paraffin-embedded tissue blot (PET blot) analysis of rPrP-res inoculated mice and control CD-1 mice that went through exactly the same treatments as the mice used for FIG. 15. Collectively, rPrP-res inoculated mouse brains exhibited the classic neuropathological features of prion disease: spongiosis, astrogliosis, microgliosis, and abnormal PrP deposition.

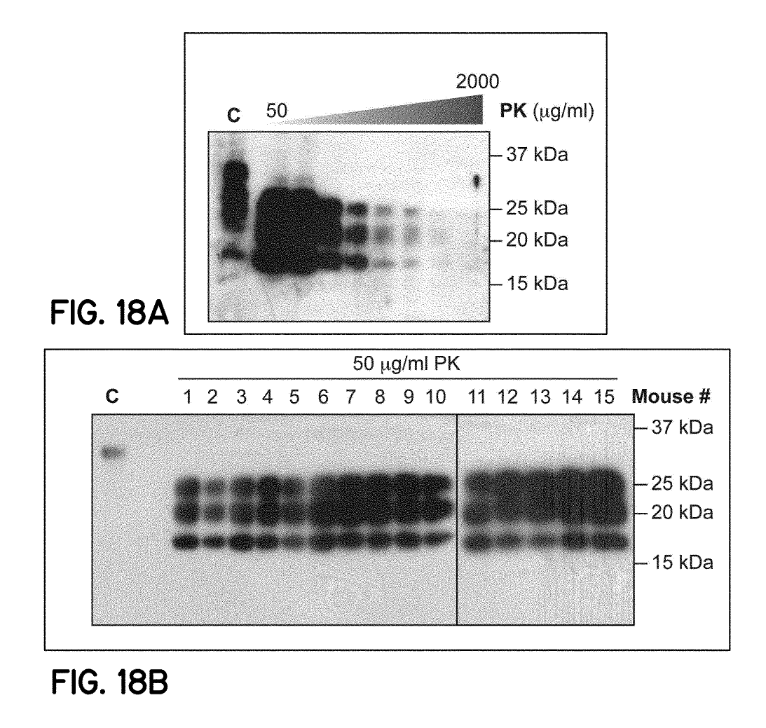

To determine whether PrP^{Path} was specifically present in rPrP-res inoculated mice, we sacrificed one mouse from each control group at 275 dpi. PrP^{Path} was immunohistochemically detected using the M20 anti-PrP antibody in rPrP-res inoculated mouse brain homogenates but not in any other control brains (FIG. 17). Histological analysis confirmed that there was no spongiosis in the control mice. The glycosylation and the electrophoretic pattern of PrP^{Path} were similar among all 15 mice (FIGS. 18A and 18B), in agreement with the similar neuropathology and relatively synchronized disease onset observed among these mice. For FIG. 18A, the brain homogenate of an rPrP-res inoculated mouse was digested with 50, 100, 250, 500, 750, 1000, 1500, or 2000 μg/ml PK at 37° C. for 1 hour and the PK-resistant PrP was detected by immunoblot analysis with POM1 anti-PrP antibody. For FIG. 18B, the PK-resistant PrP in all 15 rPrP-res injected mouse brains was detected by immunoblot analysis with POM1 anti-PrP antibody. The control (C) was the undigested brain homogenate.

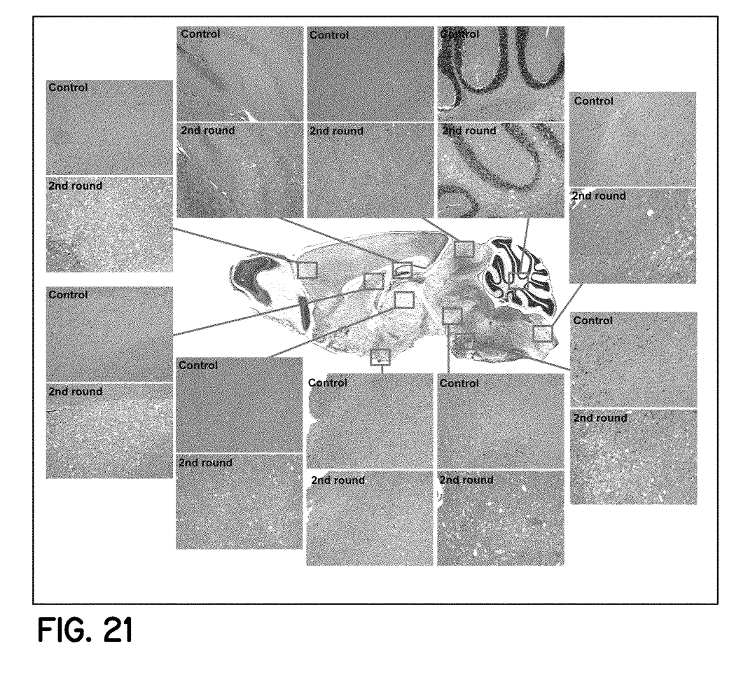

To determine whether the rPrP-res induced disease could be serially transmitted, 1% brain homogenates were prepared from 6 diseased mice and each sample was inoculated intracerebrally into 4 or 5 wild-type CD-1 mice. Around 130 dpi, all mice (n=29) developed disease and the behavior phenotypes were essentially the same as those of the rPrP-res inoculated mice. These mice reached the terminal stage of disease at 151-180 dpi and the average survival time was 166±1.5 days (FIG. 19). The marginal increase in the survival time of second round transmission could be due to the reported variation among inoculation experiments, or the influence of other components in the brain homogenate used in second round transmission. Nonetheless, PrP^{Path} was immunohistochemically detected with the POM1 antibody in all groups of mice inoculated with diseased mouse brain homogenates, but not in control mice (FIG. 20). The spongiosis pattern as determined by examination of hematoxylin and eosin stained brain regions of CD-1 mice inoculated with brain homogenates prepared from rPrP-res-infected mice (2^{nd} round) remained similar to that of rPrP-res inoculated mice (FIG. 21). Thus, similar to natural prion disease, the rPrP-res caused disease can be serially transmitted.

Inadvertent contamination is always a concern for PMCA. The only naturally occurring prion used in our lab was the RML strain, which was used only three times in our failed attempts to convert rPrP. During the last two years while we were working with rPrP-res, absolutely no naturally occurring prion was used. Our latest de novo rPrP-res formation (FIG. 4) was achieved in a new sonicator and the substrate was prepared in a lab that has never been exposed to prion. Furthermore, both behavioral and pathological phenotypes of rPrP-res inoculated mice were clearly different from those reported for RML infected mice. Therefore, it is highly unlikely that rPrP-res formation was due to an inadvertent contamination. It is also important to note that, before the inoculum was prepared, the rPrP-res had been propagated for more than 35 rounds of PMCA. Thus, even if the initial rPrP-res formation were due to contamination, the >10³⁵ dilution ensures that rPrP was the only PrP in the inoculum (FIG. 9A). Collectively, we conclude that the disease-causing agent was rPrP-res.

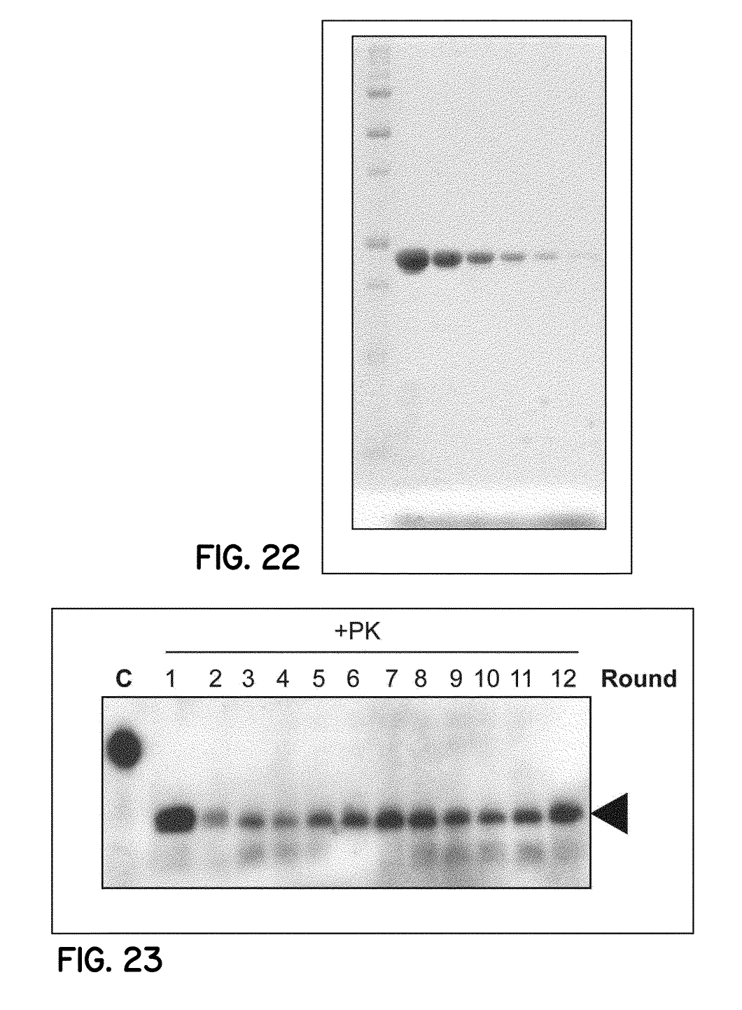

The three main components in our system were rPrP, POPG, and RNA. The purity of rPrP was verified by silver staining and rPrP was the only protein detected (FIG. 22). The mouse liver RNA was carefully chosen because PrP is not normally expressed in liver and ectopic PrP expression in the liver of PrP null mice does not support prion propagation. Because synthetic polyanions that do not encode protein can replace RNA in cell-free prion formation and propagation, the role of RNA in generating infectious prion is likely to facilitate PrP conversion rather than encoding an infectious protein. Supporting this notion, rPrP-res was successfully propagated after 12 rounds of serial PMCA using 50 μg/ml synthetic poly(A) RNA (FIG. 23), revealing that rPrP-res can be generated with virtually completely defined components. The requirement of lipid is in accordance with previous reports of higher prion infectivity in lipid membrane associated PrP^{Path}. Notably, the purified GPI-anchored PrP^{C}, which was used to produce infectious prion de novo, contained stoichiometric amounts of co-purified lipids, supporting a general role of lipid in PrP conversion.

These data demonstrate that rPrP-res was converted to a conformational state similar to the pathogenic PrP^{Path} isoform. Second, rPrP-res possessed the self-perpetuating characteristic of a prion. Third, rPrP-res caused bona fide prion disease in wild-type mice. The fact that only rPrP-res inoculated mice developed prion disease establishes that prion disease is caused by the altered conformational form of PrP.

### Methods and Materials for Example

### Reagents

Primers were ordered from Integrated DNA Technologies, Inc. RNA STAT-60 was purchased from Tel-Test, Inc. (Texas). Poly(A) RNA (polyadenylic acid) was purchased from the Midland Certified Reagent Company (Texas). Ni-NTA Agarose was purchased from Qiagen, USA. Polyvinylidene Fluoride (PVDF) membrane and ECL Plus western blotting detection reagents were purchased from GE Healthcare Life Science (New Jersey). 1-palmitoyl-2oleoyl-sn-glycero-3-phospho-(1′-sn-glycerol) (sodium salt) (POPG, 16:0-18:1 PG in chloroform) was purchased from Avanti Polar Lipids, Inc. (Alabama). Proteinase K (Lyophilizate, recombinant, PCR grade) was purchased from Roche Applied Science (Indiana). Phenylmethanesulfonyl fluoride (PMSF) was purchase from Sigma-Aldrich Inc. Guanidine Hydrochloride, Tris-HCl, Imidazole, Ethylenediaminetetraacetic acid (EDTA), NaCl, Triton X-100, SDS, β-mercaptoethanol, Isopropyl (3-D-1-thiogalactopyranoside (IPTG), 40% Acrylamide/Bis (29:1), Ammonium persulfate (APS), and N,N N′N′ tetramethylethylene diamine (TEMED) were purchased from Amresco, Inc. Other chemicals were purchased at the highest grade available from Fisher Scientific Inc. Fetal bovine serum was purchased from Thermo Scientific HyClone. Cell culture media and reagents were purchased from Invitrogen. The monoclonal POM1 anti-PrP antibody 27 was purchased from Department of Pathology, Institutes of Neuropathology, University Hospital Zurich. The monoclonal 8B4 anti-PrP antibody was a generous gift from Dr. Man-Sun Sy at Case Western Reserve University. The monoclonal SAF84 anti-PrP antibody was purchased from Cayman Chemical (Michigan). The polyclonal M20 anti-PrP antibody and the horseradish peroxidase-conjugated donkey anti-goat IgG antibody were purchased from Santa Cruz Biotechnology (California). The horseradish peroxidase-conjugated goat anti-mouse IgG antibody and the alkaline phosphatase conjugated goat anti-mouse IgG antibody were purchased from Bio-Rad Laboratories. The anti-GFAP antibody was purchased from DAKO. The anti-Iba1 antibody was purchased from Wako chemical USA. The ABC and DAB kits were purchased from Vector Lab (California). The 5-bromo 4 chloro 3-indolyl phosphate/nitroblue tetrazolium (BCIP/NBT) was purchased from Pierce (Illinois).

### Cell Culture

The SN56 mouse septal neuronal cell line was a generous gift from Dr. Bruce Wainer at Emory University. SN56 cells were maintained in OptiMEM supplemented with 10% fetal bovine serum and penicillin-streptomycin. Confluent SN56 cells were subjected to 1:10 split for continuing culture.

Expression and Purification of rPrP

Generation of rPrP Expressing Construct

The coding sequence of murine PrP23-230 was amplified by PCR and ligated into pPROEX-HTb (Invitrogen) expression vector using BamHI and HindIII restriction sites. The QuikChange site-directed mutagenesis kit (Stratagene, California) was used to eliminate the 3 amino acids in front of the coding sequence of mouse PrP23-230, which produced a construct with only one amino acid, glycine, remained at the N-terminus of recombinant PrP23-230 after the cleavage of 6-Histidine tag by tobacco etch virus (TEV) protease.

Primers for amplification of murine PrP23-230:
[SEQ. ID. NO. 1]
5′ AAAGGATCCAAAAAGCGGCCAAAGCCTGGA 3′
[SEQ. ID. NO. 2]
5′ CCCAAGCTTAGGATCTTCTCCCGTCGTAATA 3′
Primers for mutagenesis:
[SEQ. ID. NO. 3]
5′ GAAAACCTGTATTTTCAGGGCAAAAAGCGGCCAAAGCCTGG 3′
[SEQ. ID. NO. 4]
5′ CCAGGCTTTGGCCGCTTTTTGCCCTGAAAATACAAGTTTTC 3′

Purification of rPrP

The purification of rPrP was performed according to known procedures well known to those of ordinary skill in the art except that TEV protease was used to cleave the 6-Histidine tag. For TEV cleavage, the his-tagged rPrP in deionized H₂O was stirred in a beaker and 10× TEV reaction buffer (0.5 M Tris-HCl pH 8.0, 5 mM EDTA) was added drop by drop, which was followed by dropwise addition of recombinant TEV protease (in a storage buffer containing 25 mM NaPO₄, 200 mM NaCl, 2 mM EDTA, 1 mM 2-mercaptoethanol, 10% glycerol, pH 8.0). The reaction mixture was centrifuged at 2,000 rpm for 10 minutes and then incubated at 30° C. overnight. The TEV protease cleavage was assessed by SDS-PAGE and silver stain. When the cleavage was completed, the reaction mixture (carefully avoid the pellet at bottom of the tube) was loaded on a CM sepharose (Sigma) column and the rest of the purification steps were performed using standardized procedures well known to those of ordinary skill in the art.

The concentration of rPrP was calculated using the ∈280 molar extinction coefficient of 63,370 for mouse PrP23-230, according to the ExPASy Protein Server at http://us.expasy.org/tools/protparam.html. (See FIG. 17.)

### Preparation of POPG Lipid Vesicles by Sonication

POPG in chloroform was dried under a stream of nitrogen at 42° C. and then hydrated in 20 mM Tris-HCl buffer (pH 7.4) to reach a final concentration of 2.5 mg/ml. Hydrated lipids were vortexed and sonicated in a cup-hold sonicator (Misonix Inc., model XL2020) until clear. The lipid vesicles were kept under argon at 4° C.

Isolation of Total RNA from Mouse Liver

C57B1/6 or FVB/n mouse liver RNA was isolated with RNA STAT-60 (Tel-Test, Inc.) according to manufacturer's protocol. Briefly, 100 mg of mouse liver was homogenized in 1 ml RNA STAT-60 reagent, extracted by 0.2 ml chloroform, precipitated by 0.5 ml isopropanol, and dissolved in 100 μl RNase-free H₂O.

PMCA

### Preparation of PMCA Substrate

After centrifugation at 100,000 g for 1 hour at 4° C., 180 μl of soluble rPrP (0.25 mg/ml in deionized H₂O) was mixed with 16 μl of POPG (2.5 mg/ml in 20 mM Tris-HCl, pH 7.4) in a 1.5 ml siliconized microcentrifuge tube (Midsci, St. Louis) and incubated at room temperature for 10 minutes. During the incubation, a mixture of 7359 μl deionized H₂O, 500 μl of 5% Triton X-100 and 900 μl of 10× TN buffer (1.5M NaCl, 100 mM Tris-HCl, pH 7.5) was prepared in a 15 ml centrifuge tube (GeneMate, ISC BioExpress). The rPrP-POPG mixture was transferred to the 15 ml centrifuge tube, thoroughly mixed and incubated at room temperature for 5 minutes. After the incubation, 45 μl of mouse liver RNA (6 mg/ml in RNase free H₂O) was added and the substrate mixture was thoroughly mixed, aliquoted (90 μl per tube) and stored at −80° C.

### PMCA Instrument Setup

A Misonix sonicator 3000 with a microplate horn (Misonix) was used for PMCA. All reactions were carried out in 8-strip thin wall 200 μl PCR tubes that were placed in a rack in the microplate horn. The bottom of tubes were ˜3 mm above the horn surface. The sonicator was connected to an Isotemp Refrigerated Circulator (Model 910, Fisher Scientific) and the level of circulating water in the horn was adjusted to cover the reaction mixture in the PCR tubes. Water temperature is set at 39° C. in the circulator so that the temperature of water bath in the microplate horn is at 37° C. During the reaction, the microplate horn was covered with a plastic wrap to prevent evaporation. Sonicator is programmed to sonicate for 30 seconds followed by a 29.5-minute incubation and the output of the sonicator was set at 6.0. For each round of PMCA, the sonication-incubation cycle repeated for 48 times (24 hours).

Serial PMCA for De Novo rPrP-res Formation

The PMCA substrate was thawed at room temperature and briefly centrifuged. For the first round PMCA, 10 μl of 1% Triton X-100 in PBS was added to the substrate. At the end of first round, the reaction product was mixed by pipetting up and down 20 times and 10 μl reaction product was transferred to a new tube containing 90 μl substrate for the second PMCA round. The same transferring steps were repeated to carry out serial PMCA reactions (as shown in FIG. 1).

Serial PMCA for rPrP-res Propagation

In these reactions, 10 μl product of previous PMCA reaction product was added to a new tube containing 90 μl substrate and the PMCA condition was exactly the same as that of de novo rPrP-res formation.

Serial PMCA with Normal Mouse Brain Homogenate

Normal mouse brain homogenate was prepared using standard procedures well known to those of ordinary skill in the art and 50 μl aliquots were stored at −80° C. Before each reaction, the 50 p1 aliquot was adjusted to 90 μl with PBS (without Mg²⁺ or Ca²⁺, Invitrogen) and stock Triton X-100 solution. The final Triton X-100 concentration was 0.5%. In reactions without rPrP-res seed, 10 μl PBS was added to the substrate. The PMCA reaction was carried out exactly as that of rPrP PMCA.

### PK Digestion of PMCA Products

PK Digestion of rPrP after PMCA

After each round of PMCA, 30 μl PMCA product from each sample was transferred to a 1.5 ml microcentrifuge tube, respectively. Ten microliters of stock PK solution (100 μg/ml) was added to 30 μl PMCA product and the final PK concentration was 25 μg/ml. For rPrP PMCA products, the PK digestion was carried out at 37° C. for 30 minutes. The digestion was terminated by adding PMSF (5 mM final concentration) and incubating samples on ice for 5 minutes. After incubation, 20 μg Bovine Serum Albumin was added to each sample, followed by adding 200 μl-20° C. methanol and incubating at −20° C. for 45 minutes. The samples were centrifuged at 16,300 g for 15 minutes and the pellets were resuspended in 20 μl of 1× SDS sample buffer (65 mM Tris-HCl, pH 6.8, 5% SDS, 3% mercaptoethanol, 10% glycerol and trace amount of Bromophenol Blue). After boiling for 10 minutes, samples were separated in 14% SDS-PAGE followed by immunoblot analysis.

PK Digestion of PMCA Products with Normal Mouse Brain Homogenate as Substrate

The digestion was carried out in the same manner as that of rPrP PMCA except that 10 μl of PMCA products were used for PK digestion, the final PK concentration was 100 μg/ml, the digestion was carried out at 37° C. for 1 hour, and the digested samples were directly loaded on SDS-PAGE.

Infecting SN56 Cells with rPrP-res
Preparation of rPrP-res for Infection

A 4 ml pool of PMCA product was laid on top of a 500 μl 10% (w/v) sucrose cushion (in PBS) and centrifuged at 199,000 g for 1 hour at 4° C. After removing the supernatant, the pellet was resuspended in 5 ml sterilized PBS, laid over a 500 μl 10% sucrose cushion and centrifuged at 199,000 g for 1 hour at 4° C. The pellet-washing step was repeated one more time. Pellet after second wash was resuspended in 100 μl OptiMEM, transferred to a 1.5 ml sterilized microcentrifuge tube, and sonicated in a water cooled cup-horn sonicator for 5 minutes before infection.

### Cell Infection

SN56 cells were seeded in a 24-well tissue culture plate 2 days prior to infection and were around 70% confluent on the day of infection. After removing the media, cells were washed once with OptiMEM, incubated with rPrP-res (80 μl rPrP-res plus 150 μl OptiMEM without serum) for 4 hours in a CO₂ incubator, and then 1.2 ml complete cell culture media with serum was added. The cell culture media was changed in the next day. Once confluent, cells in the 24-well plate were washed with PBS, trypsinized, and transferred to a 6-well tissue culture plate. When reached confluence in the 6-well tissue culture plate, cells were subject to a 1:10 split, which was counted as passage 2. Then, cells were cultured with routine cell culture procedures well known to those of ordinary skill in the art.

### Analysis of PK-Resistant PrP in SN56 Cells

Confluent cells in one well of a 6-well tissue culture plate were washed with ice-cold PBS and lysed on ice in 200 μl cell lysis buffer (10 mM Tris-HCl pH7.5, 150 mM NaCl, 2 mM EDTA, 0.5% Triton X-100, 0.5% sodium deoxycholate). Cell lysates were stored at −20° C. until analysis. For passage 2 analysis, collected cells were washed with ice-cold PBS and lysed in the cell lysis buffer. For PK digestion, cell lysates were sonicated in a water-cooled cup-horn sonicator for 5 minutes and 195 μl of each lysate was transferred to a new 1.5 ml microcentrifuge tube. Five microliters of 1 mg/ml PK stock solution was added to each tube and the final PK concentration was 25 μg/ml. The PK digestion was carried out by a 1-hour incubation at 37° C. For terminating the PK digestion, 10 μl 100 mM PMSF was added to each sample and the lysates were incubated on ice for 5 minutes. Then, the PK digested lysates were centrifuged at 100,000 g for 1 hour at 4° C. and the pellet was resuspended in 1× SDS-PAGE sample buffer containing 5% SDS, boiled for 10 minutes, separated in 14% SDS-PAGE and subject to immunoblot analysis.

### Intracerebral Injection

### Preparation of Inocula

PMCA reactions were carried out for 24 rounds. The presence of rPrP-res was determined by immunoblot analysis and the remaining PMCA products were stored in the −80° C. freezer. Products of PMCA were pooled together for preparation of inoculum 4. For the preparation, 5 ml of PMCA products, control #1, or control #2 (Table 1) was subjected to the same pelleting and washing treatments as described in the “Preparation of rPrP-res for cell infection”. The pellet after second wash was resuspended in 625 μl of inoculation diluent (PBS containing 1 mg/ml BSA). For preparation of inoculum 3 (Table 1), 67 μl of rPrP (0.25 mg/ml in ddH₂O), 6 μl POPG (2.5 mg/ml in 20 mM Tris-HCl pH 7.4), 17 μl of mouse liver total RNA (6 mg/ml in ddH₂O), 10 μl 10 XPBS, 350 μl 1 XPBS, and 1.5 μl BSA (30%, Sigma) were mixed in a sterilized microcentrifuge tube and used directly for injection.

### Intracerebral Injection

Each 8-week-old female CD-1 mouse (Harlan) was inoculated intracerebrally with 30 μl inoculum. To avoid cross contamination, each inoculum was injected on different days in the biosafety cabinet. The injection was carried out using 1-ml disposable syringes with 25-gauge disposable needles. The injection site was in the right parietal lobe, which was 1 mm right to the sagittal suture and 1 mm anterior to the lambdoid suture.

### Detection of PrP in the Inocula

After intracerebral injection, 30 μl of the remaining inocula were subjected to 25 μg/ml PK digestion at 37° C. for 1 hour, respectively. The PK digested inocula were centrifuged at 100,000 g for 1 hour at 4° C. The pellets (PK-digested inocula) and 1 μl of each undigested inoculum (undigested inocula) were subjected to 14% SDS-PAGE followed by immunoblot analysis.

### PK Digestion of Mouse Brain Homogenate

Terminally diseased mice were sacrificed and brains were dissected sagittally. Half of the mouse brain was used for biochemical analysis, which was homogenized in PBS without Mg²⁺ or Ca²⁺ (Invitrogen) to prepare for 10% (w/v) mouse brain homogenate. Stock Triton X-100 and sodium deoxycholate solutions were added to an aliquot of the brain homogenate to reach final concentrations of 0.5% Triton X-100 and 0.5% sodium deoxycholate. The detergent lysates were vortexed briefly and incubated on ice for 10 minutes. For PK-digestion, 20 μg of detergent lysate was subjected to PK digestion at 37° C. for 1 hour and the reaction was terminated by adding PMSF followed by a 5-minute incubation on ice. The concentration of PK was indicated in the figure legend.

Immunoblot Analysis

Samples separated by SDS-PAGE were transferred to poly(vinylidene difluoride) membrane (GE Healthcare) for immunoblot analyses. Blots were blocked by 5% nonfat milk in tris-buffered saline buffer (TBST) with 0.1% Tween-20 (50 mM Tris-HCl, 150 mM NaCl, pH 8.0, 0.1% Tween-20), incubated with primary and secondary antibodies successively, and developed by ECL-plus reagent (GE Healthcare). The following antibodies were used: monoclonal POM1 anti-PrP antibody at 1:2500, monoclonal 8B4 anti-PrP antibody at 1:5000, M20 anti-PrP polyclonal antibody (Santa Cruz Biotechnology) at 1:200, horseradish peroxidase-conjugated goat anti-mouse IgG antibody (Bio-Rad Laboratories) at 1:5000, and horseradish peroxidase-conjugated donkey anti-goat IgG antibody (Santa Cruz Biotechnology) at 1:5000. FIG. 2 shows the detection result of a rPrP conversion. The PMCA products were subjected to 25 mcg/ml PK digestion, and the PK-resistant rPrP was detected by immunoblot analysis with POM1 antibody. In FIGS. 2 and 6 represents undigested rPrP as a control. Further, FIG. 3 shows serial transmissibility of converted rPrP. The undigested ninteenth and twentieth round PMCA products shown in FIG. 2 were pooled together and used to seed eight reactions for another PMCA round. The PK-resistant PrP was detected by immunoblot analysis with POM1 anti-PrP antibody. In FIG. 3, C is undigested rec-PrP as a control.

### Second Round Injection

The 10% (w/v) mouse brain homogenates were diluted in inoculation diluent (PBS plus BSA) to produce inocula containing 1% (w/v) mouse brain homogenate and 1 mg/ml BSA. Each 8-week-old female CD-1 mouse (Harlan) received 30 μl intracerebral injection.

### Histopathological Analyses

### Tissue Fixing, Decontamination and Processing

Sagittally dissected half brains were fixed by immersion in 4% formaldehyde (in PBS) at 4° C. for 2 days, decontaminated by incubating in 88% formic acid for 1-1.5 hours, and followed by immersion in 4% formaldehyde (in PBS) at 4° C. for 1-5 days. Afterwards, the brains then were dehydrated and paraffin embedded. Five-μm-thick sections were stained with Harris Hematoxylin (Sigma) and Eosin Y (Fisher).

### Immunohistochemical Staining

For immunohistochemical staining of astroglia (GFAP) and microglia (Iba1), 5-μm-thick sections were deparaffinized, rehydrated, and subjected to antigen retrieval by using the epitope unmasking solution (ProHisto). An antibody amplifer system (ProHisto) was used according to manufacturer recommended protocol to enhance the quality of immunohisto-chemical detection. The primary antibodies were anti-GFAP antibody (DAKO, 1:4000) and anti-Iba1 (Wako, 1:2500). ABC and DAB kits were purchased from Vector Lab and used according to manufacturer recommended protocol.

For PrP immunohistochemical staining, 5-μm-thick sections were deparaffinized, rehydrated, and incubated in 88% formic acid for 5 minutes at room temperature. Sections were washed twice in ddH₂O for 5 minutes each, autoclaved at 121° C. for 12 minutes, and cooled down on bench for ˜30 minutes (until the temperature was lower than 42° C.). The staining procedure was the same as described above except that the primary antibody was SAF84 anti-PrP antibody (Cayman Chemical, 1:500).

### PET Blot

The PET blot was performed as previously described 30 with a few modifications. Briefly, 4-μm thick paraffin sections were collected onto 0.45-μm nitrocellulose membranes (Bio-Rad Laboratories) and incubated at 55° C. for 20 hours. Membranes were dewaxed by immersion in xylene (45° C., 20 minutes) and rinsed in isopropanol (2×10 minutes) followed by stepwise rehydration. After washing with TBST, the membrane was subjected to 250 μg/ml PK digestion in a buffer consisting of 10 mM Tris-HCl, pH 7.8, 100 mM NaCl, 0.1% Brij 35 for 16 hours at 55° C. After washing with TBST, the membrane was treated with 4 M guanidine thiocyanate for 10 minutes and washed 3 times in TBST. The membrane was blocked by 2% non-fat milk in TBST for 1 hour, incubated with monoclonal SAF84 anti-PrP antibody (Cayman Chemical, 1:2000 in blocking solution) for 90 minutes at room temperature, washed 3 times in TBST, and incubated with a alkaline phosphatase conjugated goat anti-mouse IgG antibody (Bio-Rad Laboratories, 1:3000 in blocking solution) for 1 hour at room temperature. After three washes with TBST, the color was developed by 5-bromo 4 chloro 3-indolyl phosphate/nitroblue tetrazolium (BCIP/NBT, Pierce), and the images were assessed using a stereomicroscope (Leica).

While the present invention has been disclosed by reference to the details of preferred embodiments of the invention, it is to be understood that the disclosure is intended as an illustrative rather than in a limiting sense, as it is contemplated that modifications will readily occur to those skilled in the art, within the spirit of the invention and the scope of the amended claims.

What is claimed:

1. A composition for converting a non-pathogenic prion protein (“PrP^{N}”) into a prion protein in a pathogenic conformation (“PrP^{Path}”) comprising a lipid and/or a polyanion at a concentration sufficient to promote the conversion of the PrP^{N} into the PrP^{Path}.

2. The composition of claim 1 wherein the lipid is at least one of an anionic lipid, a cationic lipid, a zwitterionic lipid, or a neutral lipid.

3. The composition of claim 1 wherein the lipid is a phospholipid.

4. The composition of claim 1 wherein the lipid is a synthetic lipid.

5. The composition of claim 4 wherein the synthetic lipid is a synthetic anionic phospholipid.

6. The composition of claim 5 wherein the synthetic anionic phospholipid is at least one of a 1-palmitoyl-2-oleoylphosphatidylglycerol(“POPG”), 1-palmitoyl-2-oleoyl-sn-glycero-3-phosphocholine (“POPC”), 1-palmitoyl-2-arachidonoyl-sn-glycero-3-phosphocholine (“PAPC”), 1-palmitoyl-2-arachidonoyl-sn-glycero-3-phosphoglycerol (“PAPG”) or lipids extracted from a tissue.

7. The composition of claim 1 wherein the polyanion is a nucleic acid.

8. The composition of claim 7 wherein the nucleic acid is a ribonucleic acid.

9. The composition of claim 1 further comprising a buffer.

10. The composition of claim 9 wherein the buffer includes at least a detergent.

11. A method for the converting a PrP^{N} into a PrP^{Path}comprising:
mixing a PrP^{N} with a conversion substrate wherein the conversion substrate includes a lipid and/or a polyanion,
conducting at least one round of protein misfolding cyclic amplification (“PMCA”) on the mixture of the PrP^{N} and the conversion substrate wherein the at least one round of PMCA includes at least one sonication-incubation cycle that includes a period of sonication followed by a period of incubuation.

12. The method of claim 10 wherein the period of incubation is longer than the period of sonication.

13. The method of claim 10 wherein the at least one round includes at least 15 sonication-incubation cycles.

14. The method of claim 10 wherein the PrP^{N} is a recombinant non-pathogenic prion protein.

15. A method of identifying a potential therapeutic substance affecting the a PrP^{Path} comprising:
mixing a potential therapeutic substance with a PrP^{N} and a conversion substrate wherein the conversion substrate includes a lipid and a polyanion;
conducting at least one round of PMCA on the mixture of the potential therapeutic substance, the PrP^{N} and the conversion substrate; and
evaluating the mixture to determine if the potential therapeutic substance affected the conversion of the PrP^{N} into a PrP^{Path}.

16. The method of claim 15 wherein evaluating the mixture includes at least one of determining the presence of PrP^{Path}, the conversion rate of PrP^{N} to PrP^{Path}, comparing the results obtained with the mixture including the potential therapeutic substance with a control mixture lacking the potential therapeutic substance.

17. The method of claim 15 wherein the mixture of the potential therapeutic substance with the PrP^{N} and conversion substrate is seeded with a PrP^{Path}.

18. The method of claim 15 wherein the PrP^{N} is a recombinant non-pathogenic prion protein.

19. A method of diagnosing PrP^{Path} infection in a subject comprising:
mixing a sample of tissue, blood, or body fluid from a subject with a PrP^{N} and a conversion substrate wherein the conversion substrate includes a lipid and a polyanion;
conducting at least one round of PMCA on the mixture of the sample, the PrP^{N} and the conversion substrate; and
evaluating the mixture for the presence of PrP^{Path}.

20. The method of claim 19 wherein the PrPN is a recombinant non-pathogenic prion protein.
